(19) United States
(12) Patent Application Publication
(10) Pub. No.: US 2024/0123120 A1
(43) Pub. Date: Apr. 18, 2024

(54) HIGH-ADHESION ARTIFICIAL CORNEAL ENDOTHELIAL SHEET, PREPARATION METHOD AND USE THEREOF

(71) Applicant: Eye Institute of Shandong First Medical University, Qingdao, Shandong (CN)

(72) Inventors: Weiyun SHI, Qingdao,Shandong (CN); Ting WANG, Qingdao,Shandong (CN); Qingjun ZHOU, Qingdao,Shandong (CN); Xiaoyu ZHANG, Qingdao,Shandong (CN); Hongwei WANG, Qingdao,Shandong (CN)

(21) Appl. No.: 18/041,954

(22) PCT Filed: Jul. 13, 2022
(86) PCT No.: PCT/CN2022/105454

§ 371 Date: Feb. 16, 2023

(51) Int. Cl. A61L 27/36; A61L 27/16; A61L 27/52

(52) U.S. Cl. CPC A61L 27/3604; A61L 27/16; A61L 27/3687; A61L 27/52; A61L 2430/16

(57) ABSTRACT

A corneal implant with adhesion-increase treatment is associated with a method for increasing the adhesion of the artificial corneal implant by an epoxy-amine ring-opening reaction of the polyamino high-molecular polymer and glycidyl methacrylate to obtain a methacrylate modified polyamino polymer. The method improves the adhesion and biocompatibility of the artificial corneal endothelial sheet by photo-initiated free radical polymerization reaction, and has a high oxygen permeability and a high adhesion. It can be used for treating patients with corneal endothelial decompensation, playing a barrier role to block aqueous humor, eliminating corneal edema, and reducing the risk of corneal graft detachment after surgery.

### TECHNICAL FIELD

The present invention relates to a corneal implant with adhesion-increase treatment and a method for increasing the adhesion of an artificial corneal endothelial sheet, which belongs to the field of medical artificial replacement materials.

### BACKGROUND

The corneal endothelial layer is the innermost layer of the cornea, and its structural and functional integrity are important factors for maintaining the normal physiological metabolism of the cornea. Decreased endothelial cell density and endothelial pump dysfunction can lead to corneal endothelial decompensation, manifested as corneal edema and opacity, which seriously affects the vision of patients, rendering them in obvious eye pain and reduces life quality. Corneal endothelial decompensation is one of the common complications postoperatively in inner eye surgeries such as cataract and etc, and also the terminal manifestation of primary diseases such as corneal endothelial dystrophy. Initially, corneal endothelial decompensation can only be treated with penetrating keratoplasty (PKP). With the development of surgical techniques, the proportion of endothelial keratoplasty (EK) has gradually increased, preserving the patient's own corneal stromal while transplanting only the endothelia layer of the lesion. The EK surgeries include Descemet stripping endothelial keratoplasty (DSEK) and Descemet membrane endothelial keratoplasty (DMEK), both of which are surgically difficult. They not only need fresh corneal donors, but also request high donor quality. Some patients may suffer recurrence of corneal implant endothelial decompensation due to excessive loss of corneal endothelium during surgery, and 35% of patients may go through postoperative corneal implant detachment and need one or more rebubbling procedures.

### SUMMARY OF THE INVENTION

Artificial corneal endothelial sheet refers to an artificial material attached to the surface of the posterior corneal matrix to isolate aqueous humor as a barrier, also known as corneal implants, corneal endothelial patches, etc.

Based on a large number of clinical studies, the object of the present invention is to provide an artificial corneal endothelial sheet with adhesion-increase treatment and its preparation method and application thereof. The modified artificial endothelial sheet has better adhesion to the posterior corneal matrix.

The artificial corneal endothelial sheet provided by the inventor is transparent, extremely soft, foldable, and has good compatibility with aqueous humor and no toxic side effects.

In one aspect of the present invention, a high-adhesion artificial corneal endothelial sheet is provided, wherein an adhesion-increase functional material is attached to the surface of the artificial corneal endothelial sheet after adhesion-increase treatment; the adhesion-increase treatment includes obtaining a methacrylate modified polyamino polymer by an epoxy-amine ring-opening reaction of polyamino high-molecular polymer and glycidyl methacrylate, and performing surface modification of the artificial corneal endothelial sheet by photo-initiated free radical polymerization reaction.

Preferably, the adhesion-increase treatment comprises the following steps:
Step 1) putting said adhesion-increase functional material into distilled water, stirring until dissolved, and forming a solution with a concentration of 0.08-0.14 g/mL;
Step 2) adding 1.0-2.0 mL of glycidyl methacrylate to the solution in Step 1);
Step 3) placing the mixed solution of Step 2) at 40-70° C. for 4-8 h;
Step 4) dialyzing in distilled water to remove unreacted glycidyl methacrylate and oligomer by the 12-14 kDa cut-off dialysis tube, and vacuum freeze-drying to obtain a solid white foam;
Step 5) putting 100.0-300.0 mg of solid white foam of Step 4) into 2.0-6.0 mL of distilled water and completely dissolving to obtain a mixture;
Step 6) soaking the artificial endothelial sheet in the mixture of Step 5) and adding 6.0-10.0 μL of 10% DMPA solution;
Step 7) placing the mixture of Step 6) under a UV lamp with a wavelength of 365 nm for 20-60 min, and continuously stirring the mixture during irradiation;
Step 8) washing with distilled water to obtain artificial corneal endothelial sheet with adhesion-increase treatment.

Preferably, the mass ratio of the adhesion-increase functional material, distilled water, and glycidyl methacrylate in Step 1) and Step 2) is (4-7): 50:(1-2).

Preferably, the adhesion-increase functional material is polyamino polymer, selected from one or more of gelatin, chitosan, and serum albumin.

Preferably, the material of the artificial corneal endothelial sheet is acrylate-like material, selected from one or more of hydroxyethyl methacrylate/methyl methacrylate copolymer, polymethyl methacrylate, poly(hydroxyethyl methacrylate), acrylic acid hydrogel, and methacrylic acid hydrogel. Preferably, the artificial corneal endothelial sheet has a light transmittance of 79%-85% (400-800 nm).

Another aspect of the present invention, a method of increasing the adhesion of the artificial corneal endothelial sheet is provided, wherein, comprising the following steps:
Step 1) putting said adhesion-increase functional material into distilled water, stir until dissolved, and form a solution with a concentration of 0.08-0.14 g/mL;
Step 2) adding 1.0-2.0 mL of glycidyl methacrylate to the solution in step 1);
Step 3) placing the mixed solution of Step 2) at 40-70° C. for 4-8 h;
Step 4) dialyzing in distilled water to remove unreacted glycidyl methacrylate and oligomer by the 12-14 kDa cut-off dialysis tube, and vacuum freeze-drying to obtain a solid white foam;
Step 5) putting 100.0-300.0 mg of solid white foam of Step 4) into 2.0-6.0 mL of distilled water and completely dissolve to obtain a mixture;
Step 6) soaking the artificial endothelial sheet in the mixture of Step 5) for 1-2 h and adding 6.0-10.0 μL of 10% DMPA solution;
Step 7) placing the mixture of Step 6) under a UV lamp with a wavelength of 365 nm for 20-60 min, and continuously stirring the mixture during irradiation;
Step 8) washing with distilled water to obtain artificial corneal endothelial sheet with adhesion-increase treatment.

Preferably, the mass ratio of the adhesion-increase functional material, distilled water, and glycidyl methacrylate in Step 1) and Step 2) is (4-7): 50:(1-2).

Preferably, the adhesion-increase functional material is polyamino polymer, selected from one or more of gelatin, chitosan, and serum albumin.

Preferably, the material of the artificial corneal endothelial sheet is acrylate-like material, selected from one or more of hydroxyethyl methacrylate/methyl methacrylate copolymer, polymethyl methacrylate, poly(hydroxyethyl methacrylate), acrylic acid hydrogel, and methacrylic acid hydrogel. Another aspect of the present invention, this invention provides a use of the above-described artificial corneal endothelial sheet and/or the method for increasing the adhesion of artificial corneal endothelial sheet in preparing medical instruments for relieving or treating corneal endothelial injury, corneal endothelial dysfunction, and corneal endothelial decompensation.

Another aspect of the present invention, this invention provides a use of the above-mentioned artificial corneal endothelial sheet and/or methods for increasing the adhesion of the artificial corneal endothelial sheet in preparing medical instruments for relieving or treating patients with corneal thickness abnormality, corneal transparency degradation, corneal edema, vision decline or loss, or eye dryness and pain resulting from corneal endothelial decompensation.

Preferably, the medical device is a graft, patch or kit.

### Beneficial Effects

The artificial corneal endothelial sheet provided by the present invention is a transparent, non-degradable optical material, using polyamino high-molecular polymers with less antigenicity, such as gelatin, chitosan, and serum albumin, to prepare methacrylate-modified polyamino high-molecular polymer by epoxy-amine ring-opening reaction with glycidyl methacrylate, the modification product generates a layer of hydrogel on the surface of the acrylate artificial endothelial sheet by photo-initiated free radical polymerization reaction, which can increase the adhesion of the artificial endothelial sheet to the corneal matrix, the artificial corneal endothelial sheet features strong adhesion, good biocompatibility and no toxicity, and it hardly falls off after implantation; extremely soft and easy to fold, the artificial endothelial sheet can be implanted in the anterior chamber with small incisions, needless of cell-loading, and free from considerations of cell destruction during the operation, which greatly reduces the difficulty of surgery, thereby reducing complications such as excessive loss of corneal endothelial cells and corneal graft detachment postoperatively.

The corneal implants provided by the present invention are expected to replace the traditional donor corneal endothelial grafts, reducing the number of corneal transplants.

### BRIEF DESCRIPTION OF THE DRAWINGS

FIG. 1 shows the appearance features of the artificial corneal endothelial sheet in Example 1, panels A and B show that the artificial corneal endothelial sheet is transparent and curvature in; panel C shows that, the anterior chamber is full of air and the artificial corneal endothelial sheet is in place after the surgery in experimental group 1, Example 4.

FIG. 2 shows the light transmittance of the artificial corneal endothelial sheet prepared in Example 1-3 with adhesion-increase treatment and that of normal rabbit cornea, indicating that their transparencies are similar.

FIG. 3 shows slit lamp and OCT observation diagram of postoperative rabbit cornea from blank group, control group, experimental group 1, experimental group 2, experimental group 3 in Example 4, wherein experimental group 1, experimental group 2 and experimental group 3 respectively represent animal experiment results relating to the corresponding modification methods in Examples 1-3.

FIG. 4 shows corneal HE staining diagram of Example 4, wherein panel A is an HE staining diagram of a normal cornea; and panel B is an HE staining diagram of the central cornea 2 weeks after the implantation of endothelial sheet with adhesion-increase treatment in experimental group 1, Example 4, indicating that there is no obvious inflammatory response on the endothelial surface.

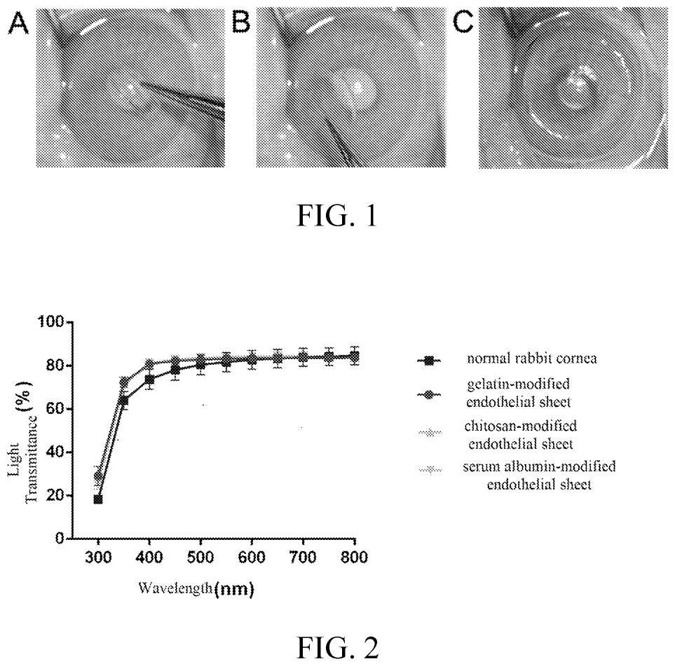

### DETAILED DESCRIPTION OF THE EMBODIMENTS

The present invention is further illustrated by the following embodiments explaining the present invention, the following embodiments are only used to illustrate the present invention and should not be regarded as limiting the scope of the present invention. Unless otherwise specified, the technical and scientific terms used herein are generally understood by those of ordinary skill in the art to which the invention belongs. If the specific conditions are not indicated in the embodiment, the conditions shall be carried out in accordance with the general conditions or the conditions recommended by the manufacturer. If the manufacturers of the reagents or instruments are not identified, they are all conventional products that can be commercially available.

The present invention has no special restrictions on the source of artificial corneal endothelial sheets, and the source of artificial corneal endothelial sheets well known in the art can be used. In the embodiments of the present invention, the artificial corneal endothelial sheet is self-made by Eye Research Institute of Shandong First Medical University.

Relevant parameters of the artificial corneal endothelial sheets:
1. The material of artificial corneal endothelial sheet is acrylate, which can be selected from one or more of hydroxyethyl methacrylate/methyl methacrylate copolymer, polymethyl methacrylate, poly(hydroxyethyl methacrylate), acrylic hydrogel, and methacrylic acid hydrogel.
2. Artificial corneal endothelial sheet: diameter of 5.0-7.0 mm, thickness of 25.0-70.0 μm, radius of curvature of 6.0-9.0 mm.
3. Light transmittance: the light transmittance of artificial corneal endothelial sheet gradually increases with the increase of light wavelength in the wavelength range of 300-800 nm, and the light transmittance can reach 79%-85% in the wavelength range above 400 nm.

### Example 1: Preparation of Gelatin Modified Artificial Corneal Endothelial Sheet to Increase Adhesion

(1) Add 5.0 g type A pigskin gelatin to the centrifuge tube;
(2) Add 45.0 mL of distilled water to the centrifuge tube of step (1);
(3) Stir the above mixture at 50° C. until completely dissolved to form a uniform gelatin solution;
(4) Add 1.0 mL of glycidyl methacrylate to the centrifuge tube of step (3);
(5) Place the centrifuge tube containing the mixed solution obtained in step (4) in a 50° C. water bath for 6 h;
(6) Use a 12-14 kDa cut-off dialysis tube in a large amount of distilled water to dialyze for 5 days to remove unreacted glycidyl methacrylate and oligomers;
(7) The sample obtained in step (6) is vacuum freeze-dried for 72 hours, and the obtained solid white foam is stored at 4° C. for reuse;
(8) Add 200.0 mg of solid white foam from step (7) to a centrifuge tube;
(9) Add 4.0 mL of distilled water to the centrifuge tube in step (8);
(10) Place the above centrifuge tube in 50° C. water bath for 10 h until completely dissolved;
(11) Place the artificial endothelial sheet in the above solution and soak it in a flask for 1 h;
(12) Add 8.0 μL of 10% DMPA solution to the above mixed solution;
(13) Add a magnetic rotor to the above mixed solution, with running the rotor by a magnetic stirrer, the flask with the solution and the artificial endothelial sheet in step (12) is placed under an ultraviolet lamp with a wavelength of 365 nm for 30 min;
(14) Wash at least 6 times with distilled water to obtain an artificial endothelial sheet modified by gelatinization.

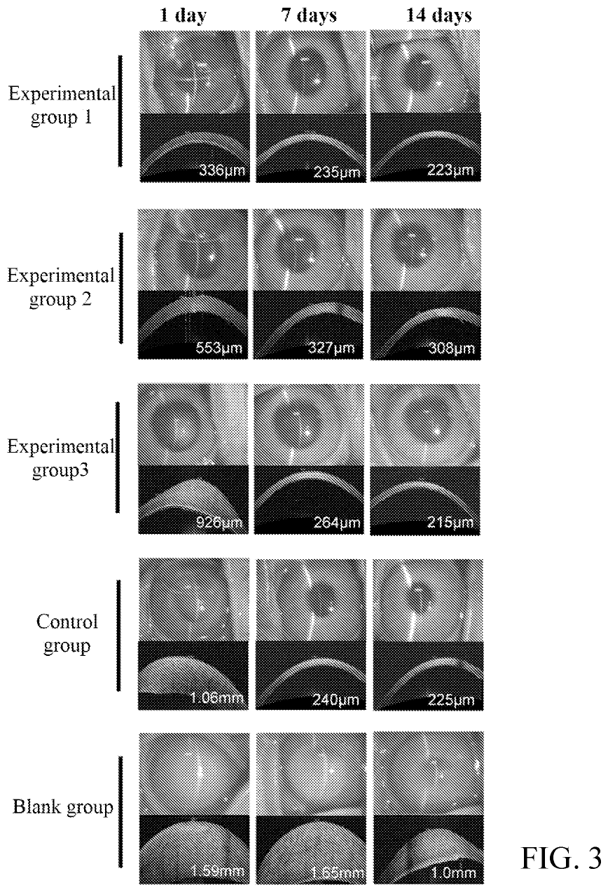

The light transmittance of gelatin-modified artificial endothelial sheet is tested by ultraviolet spectrophotometer, and the results are shown in FIG. 2. The light transmittance of the prepared gelatin-modified artificial corneal endothelial sheet is similar to that of normal rabbit cornea, indicating that both transparencies are similar.

### Example 2: Preparation of an Artificial Corneal Endothelial Sheet Modified by Chitosan to Increase Adhesion

(1) Add 4.5 g chitosan to the centrifuge tube;
(2) Add 50.0 mL of distilled water to the centrifuge tube in step (1);
(3) Stir the above mixture at 50° C. until completely dissolved to form a uniform chitosan solution;
(4) Add 1.0 mL of glycidyl methacrylate to the centrifuge tube of step (3);
(5) Place the centrifuge tube containing the mixed solution obtained in step (4) in a 50° C. water bath for 5 h;
(6) Use a 12-14 kDa cut-off dialysis tube in a large amount of distilled water to dialyze for 5 days to remove unreacted glycidyl methacrylate and oligomers;
(7) The sample obtained in step (6) is vacuum freeze-dried for 72 hours, and the obtained solid white foam is stored at 4° C. for reuse;
(8) Add 200.0 mg of solid white foam from step (7) to the centrifuge tube;
(9) Add 4.5 mL of distilled water to the centrifuge tube of step (8);
(10) Place the above centrifuge tube in a 50° C. water bath for 10 h until completely dissolved;
(11) Place the artificial endothelial sheet in the above solution and soak it in a flask for 1 h;
(12) Add 8.0 μL of 10% DMPA solution to the above mixed solution;
(13) Add a magnetic rotor to the above mixed solution, with running the rotor by a magnetic stirrer, the flask with the solution and an artificial endothelial sheet in step (12) is placed under an ultraviolet lamp with a wavelength of 365 nm for 30 min;
(14) Wash at least 6 times with distilled water to obtain an artificial endothelial sheet modified by chitosan.

The light transmittance of chitosan-modified artificial endothelial sheet is tested by ultraviolet spectrophotometer, and the results are shown in FIG. 2, and the light transmittance of the prepared chitosan-modified artificial corneal endothelial sheet is similar to that of normal rabbit cornea, indicating that both transparencies are similar.

### Example 3: Preparation of an Artificial Corneal Endothelial Sheet Modified with Serum Albumin to Increase Adhesion

(1) Add 6.0 g of serum albumin to the centrifuge tube;
(2) Add 40.0 mL of distilled water to the centrifuge tube in step (1);
(3) Stir the above mixture at 50° C. until completely dissolved to form a uniform albumin solution;
(4) Add 1.5 mL of glycidyl methacrylate to the centrifuge tube of step (3);
(5) The centrifuge tube containing the mixed solution obtained in step (4) is placed in a 50° C. water bath for 8 h;
(6) Use a 12-14 kDa cut-off dialysis tube in a large amount of distilled water to dialyze for 5 days to remove unreacted glycidyl methacrylate and oligomers;
(7) The sample obtained in step (6) is vacuum freeze-dried for 72 hours, and the obtained solid white foam is stored at 4° C. for reuse;
(8) Add 250.0 mg of solid white foam from step (7) to the centrifuge tube;
(9) Add 4.0 mL of distilled water to the centrifuge tube in step (8);
(10) Place the above centrifuge tube in a 50° C. water bath for 10 h until completely dissolved;
(11) Place the artificial endothelial sheet in the above solution and soak in a flask for 2 h;
(12) Add 10.0 μL of 10% DMPA solution to the above mixed solution;
(13) Add a magnetic rotor to the above mixed solution, with running the rotor by a magnetic stirrer, the flask with the solution and an artificial endothelial sheet in step (12) is placed under an ultraviolet lamp with a wavelength of 365 nm for 30 min;
(14) Wash at least 6 times with distilled water to obtain an artificial endothelial sheet modified by serum albumin.

The light transmittance of serum albumin-modified artificial endothelial sheet is tested by ultraviolet spectrophotometer, and the results are shown in FIG. 2, and the light transmittance of the prepared serum albumin-modified artificial corneal endothelial sheet is similar to that of normal rabbit cornea, indicating that both transparencies are similar.

### Example 4: Animal Experiments

### 1. Materials and Methods

### 1.1 Laboratory Animals

25 New Zealand white rabbits, weighing 3.0-3.5 Kg, male rabbits.

### 1.2 Grouping

25 New Zealand white rabbits are randomly divided into 5 groups, 5 in each group.
(1) Blank group: the central area of the rabbit corneal endothelium is peeled off, and no artificial corneal endothelial sheet is implanted.
(2) Control group: the central area of the rabbit corneal endothelium is peeled off, and an artificial corneal endothelial sheet without adhesion-increase treatment is implanted (the diameter of the endothelial sheet is 6.5 mm, the thickness is 50.0 μm, and the radius of curvature is 7.32 mm).
(3) Experimental group 1: the central area of the rabbit corneal endothelium is peeled off, and an artificial corneal endothelial sheet modified by gelatin in Example 1 is implanted (the diameter of the endothelial sheet is 6.5 mm, the thickness is 50.0 μm, and the radius of curvature is 7.32 mm).
(4) Experimental group 2: the central area of the rabbit corneal endothelium is peeled off, and an artificial corneal endothelial sheet modified by chitosan in Example 2 is implanted (the diameter of the endothelial sheet is 6.5 mm, the thickness is 50.0 μm, and the radius of curvature is 7.32 mm).
(5) Experimental group 3: the central area of the rabbit corneal endothelium is peeled off, and an artificial corneal endothelial sheet modified by serum albumin in Example 3 is implanted (the diameter of the endothelial sheet is 6.5 mm, the thickness is 50.0 μm, and the radius of curvature is 7.32 mm).

### 1.3 Establishment of Animal Models of Corneal Endothelial Decompensation

(1) Anesthetize New Zealand white rabbit by intravenously injecting 25.0 mg/kg pentobarbital sodium at the ear margin.
(2) Drop 0.5% proxymetacaine hydrochloride on the surface of the rabbit cornea for topical analgesia.
(3) Rinse the conjunctival sac with 0.9% sodium chloride injection, and use a cotton swab to fully clean the ocular surface.
(4) Use a 6.5 mm ring drill to make an imprint on the center of the rabbit corneal epithelium.
(5) Use a 15° piercing knife to make a puncture opening at 12 o'clock spot at the corneal limbus, inject 0.02 mg/mL carbachol injection to induce miosis, and inject viscoelastic agent to form anterior chamber.
(6) Use a 1 ml syringe needle and lens alignment hook to remove the endothelium marked in the center of the cornea.
(7) Use 0.9% sodium chloride injection to rinse the anterior chamber and replace the viscoelastic agent.
(8) Inject 4.0 mg/mL gentamicin injection and heparin sodium injection into the anterior chamber sequentially to reduce the inflammatory response of the anterior chamber.
(9) Suture the incision of the rabbits in blank group and use 0.9% sodium chloride injection to form the anterior chamber.
(10) Re-injected with viscoelastic agent to form the anterior chamber of the rabbits in the control group and the experimental groups; implant an artificial endothelial sheet without adhesion-increase treatment in control group; implant an artificial endothelial sheet with gelatin-modified prepared in Example 1 in the experimental group 1; implant an artificial endothelial sheet with chitosan-modified prepared in Example 2 in the experimental group 2, and implant an artificial endothelial sheet modified with serum albumin-modified prepared in Example 3 in the experimental group 3.
(11) fold the artificial corneal endothelial sheet and place into the anterior chamber using toothless forceps, and fix the corneal implant on the endothelium in the scraped central area using the alignment hook.
(12) Use 0.9% sodium chloride injection to rinse the anterior chamber again and replace the viscoelastic agent.
(13) Suture the incision with 10-0 sutures and infuse sterile air into the anterior chamber using an insulin needle.

### 2. Results (See Table 1)

### 2.1 Shedding Rate of Artificial Corneal Endothelial Sheet

On the first postoperative day, in the control group 5 rabbits have postoperative graft detachment with an incidence of 100%; in experimental group 1 one rabbit has postoperative graft detachment with an incidence of 20%; in experimental group 2 one rabbit has postoperative graft detachment with an incidence of 20%; in experimental group 3 two rabbits have postoperative graft detachment with an incidence of 40%.

### 2.2 Corneal Edema

Use a slit lamp for ophthalmological examination (FIG. 3), general pictures of the eyes are taken on days 1, 7, and 14 after surgery. The general photographs of the eyes show that on the first day after surgery, all the rabbits in the control group have artificial endothelial sheet detachment (100%), and the anterior chamber air injection is required again after surgery, the detachment proportion of artificial corneal endothelial sheet in the experimental groups are as follows, the experimental group 1 is 20%, the experimental group 2 is 20%, the experimental group 3 is 40%, rabbits' corneas in the experimental groups and the control group completely recover and are transparent within 1 week after surgery, while in the blank group corneal edema and opacity are prominent, proving that the artificial corneal endothelial sheet plays a role as barrier in blocking aqueous humor, the artificial endothelial sheet adheres well to the corneal posterior matrix after adhesion-increase treatment, reducing the risk of postoperative graft detachment.

### 2.3 Adhesion of Artificial Corneal Endothelial Sheet

Use anterior segment OCT (see Table 1, FIG. 3) to measure the thickness of the central cornea on days 1, 7, and 14 postoperatively. Corneal OCT shows that on the first day after surgery, all the rabbits in the control group have artificial endothelial sheet detachment (100%), the endothelial sheet normally falls to the lower anterior chamber after detachment. The proportions of artificial corneal endothelial sheet detachment in the experimental groups are as follows, the experimental group 1 is 20%, the experimental group 2 is 20%, and the experimental group 3 is 40%, the experimental groups and the control group return to normal thickness within 1 week after surgery, the average corneal thickness of experimental group 1 is (223±16) and the average corneal thickness of experimental group 2 is (278±44) and the average corneal thickness of the experimental group 3 is (242±19) and the average corneal thickness of the control group is (227±13) while the corneal thickness of the blank group is about (1643±50) μm within 1 week after surgery. Moreover, the experimental groups and the control group could maintain corneal transparency for 14 days after surgery. It is proved that the artificial corneal endothelial sheet after adhesion-increase treatment adheres well to the corneal matrix, and act as a barrier to block aqueous humor.

TABLE 1
Treatment and results of implanted modified
and unmodified artificial endothelial sheets

| | | Detachment | | Corneal | Corneal |
|---|---|---|---|---|---|
| | | rate of | Corneal | thickness | thickness |
| | | Corneal | transparency | (μm) | (μm) |
| Group | Treatment | transplant | (7 days) | (7 days) | (14 days) |
| Blank group | only peel off | / | edema | 1643 ± 50 | 940 ± 186 |
| | endothelium | | | | |
| Control group | implant unmodified | 100% | transparent | 227 ± 13^{a} | 238 ± 23 ^{a} |
| | endothelial sheet | | | | |
| Experimental | implant gelatin- | 20% | transparent | 223 ± 16^{a} | 213 ± 13 ^{a} |
| group 1 | modified | | | | |
| | endothelial sheet | | | | |
| Experimental | implant chitosan- | 20% | transparent | 278 ± 44^{a} | 263 ± 39^{a} |
| group 2 | modified | | | | |
| | endothelial sheet | | | | |
| Experimental | implant serum | 40% | transparent | 242 ± 19^{a} | 219 ± 9^{a} |
| group 3 | albumin-modified | | | | |
| | endothelial sheet | | | | |

^{a}means comparing with the blank group, P < 0.05; and there is no significant difference between the experimental groups and the control group.

### 2.4 Biocompatibility of Artificial Corneal Endothelial Sheet

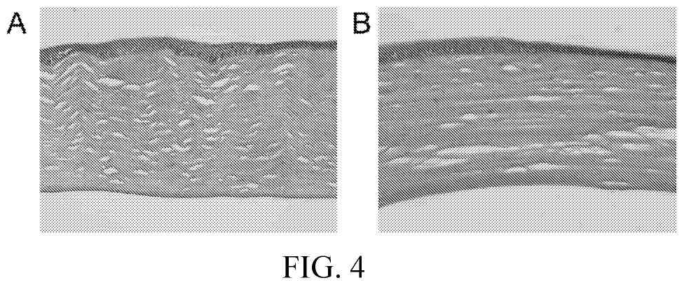

On the 14th postoperative day, the New Zealand rabbits are euthanized, the rabbits' corneas are removed for histopathological examination, and HE staining. As shown in FIG. 4, wherein panel A is the HE staining diagram of normal rabbit cornea; panel B is the HE staining diagram of the central cornea, 14th day after endothelial sheet implantation in experimental group 1, Example 4, indicating that there is no obvious inflammatory response on the endothelial surface, and the implant has good biocompatibility.

Although the specific embodiments of the present invention have been described in detail, those skilled in the art could understand. According to all the teachings that have been disclosed, various modifications and substitutions may be made to those details, which are within the protection scope of the present invention. The full scope of the invention is given by the dependent claims and any equivalents thereof.

1. A high-adhesion artificial corneal endothelial sheet, wherein a material having an adhesion-increase function is attached with the surface of said artificial corneal endothelial sheet after adhesion-increase treatment; said adhesion-increase treatment includes obtaining a methacrylate functionalized modified polyamino high-molecular polymer by an epoxy-amine ring-opening reaction of polyamino polymer and glycidyl methacrylate, and performing surface modification of said artificial corneal endothelial sheet by photo-initiated free radical polymerization reaction.

2. The high-adhesion artificial corneal endothelial sheet according to claim 1, wherein said adhesion-increase treatment comprises the following steps:
Step 1) putting an adhesion-increase functional material into distilled water, stirring until dissolved, and forming a solution with a concentration of 0.08-0.14 g/mL;
Step 2) adding 1.0-2.0 mL of glycidyl methacrylate to the solution in Step 1);
Step 3) placing the mixed solution of Step 2) at 40-70° C. for 4-8 h;
Step 4) dialyzing in distilled water to remove unreacted glycidyl methacrylate and oligomer by the 12-14 kDa cut-off dialysis tube, and vacuum freeze-drying to obtain solid white foam;
Step 5) putting 100.0-300.0 mg of solid white foam of Step 4) into 2.0-6.0 mL of distilled water and completely dissolving to obtain a mixture;
Step 6) soaking the artificial endothelial sheet in the mixture of Step 5) and adding 6.0-10.0 μL of 10% DMPA solution;
Step 7) placing the mixture of Step 6) under a UV lamp with a wavelength of 365 nm for 20-60 min, and continuously stirring the mixture during irradiation;
Step 8) washing with distilled water to obtain artificial corneal endothelial sheet with adhesion-increase treatment.

3. The high-adhesion artificial corneal endothelial sheet according to claim 2, wherein the mass ratio of said adhesion-increase functional material, distilled water, glycidyl methacrylate in Step 1) and Step 2) is (4-7): 50:(1-2).

4. The high-adhesion artificial corneal endothelial sheet according to claim 1, wherein said adhesion-increase functional material is polyamino polymer, selected from one or more of gelatin, chitosan, serum albumin.

5. The high-adhesion artificial corneal endothelial sheet according to claim 1, wherein said artificial corneal endothelial sheet is acrylate-like material, selected from one or more of hydroxyethyl methacrylate/methyl methacrylate copolymer, polymethyl methacrylate, poly(hydroxyethyl methacrylate), acrylic acid hydrogel, methacrylic acid hydrogel.

6. The high-adhesion artificial corneal endothelial sheet according to claim 1, wherein said artificial corneal endothelial sheet has a light transmittance of 79%-85% (400 nm-800 nm).

7. A method of increasing the adhesion of an artificial corneal endothelial sheet, wherein, comprises the following steps:
Step 1) putting an adhesion-increase functional material into distilled water, stirring until dissolved, and forming a solution with a concentration of 0.08-0.14 g/mL;
Step 2) adding 1.0-2.0 mL of glycidyl methacrylate to the solution in Step 1);
Step 3) placing the mixed solution of Step 2) at 40-70° C. for 4-8 h;
Step 4) dialyzing in distilled water to remove unreacted glycidyl methacrylate and oligomer by the 12-14 kDa cut-off dialysis tube, and vacuum freeze-drying to obtain solid white foam;
Step 5) putting 100.0-300.0 mg of solid white foam of Step 4) into 2.0-6.0 mL of distilled water and completely dissolving to obtain a mixture;
Step 6) soaking the artificial endothelial sheet in the mixture of Step 5) and adding 6.0-10.0 μL of 10% DMPA solution;
Step 7) placing the mixture of Step 6) under a UV lamp with a wavelength of 365 nm for 20-60 min, and continuously stirring the mixture during irradiation;
Step 8) washing with distilled water to obtain artificial corneal endothelial sheet with adhesion-increase treatment.

8. The method of increasing the adhesion of an artificial corneal endothelial sheet according to claim 7, wherein the mass ratio of said adhesion-increase functional material, distilled water, glycidyl methacrylate in Step 1) and Step 2) is (4-7): 50:(1-2).

9. The method of increasing the adhesion of an artificial corneal endothelial sheet according to claim 7, wherein said adhesion-increase functional material is polyamino polymer, selected from one or more of gelatin, chitosan, serum albumin; the material of said artificial corneal endothelial sheet is acrylate-like material, selected from one or more of hydroxyethyl methacrylate/methyl methacrylate copolymer, polymethyl methacrylate, poly(hydroxyethyl methacrylate), acrylic acid hydrogel, methacrylic acid hydrogel.

10. Use of said artificial corneal endothelial sheet according to claim 1 in preparing medical instruments for relieving or treating corneal endothelial injury, corneal endothelial dysfunction, corneal endothelial decompensation.

11. Use of said artificial corneal endothelial sheet according to claim 1 in preparing medical instruments for relieving or treating patients with corneal thickness abnormality, corneal transparency degradation, corneal edema, vision decline or loss, eye dryness and pain resulting from corneal endothelial decompensation.

12. Use according to claim 10, wherein said medical instruments are graft, patch or kit.

13. Use of said method of increasing the adhesion of an artificial corneal endothelial sheet according to claim 7 in preparing medical instruments for relieving or treating corneal endothelial injury, corneal endothelial dysfunction, corneal endothelial decompensation.

14. Use of said method of increasing the adhesion of an artificial corneal endothelial sheet according to claim 7 in preparing medical instruments for relieving or treating patients with corneal thickness abnormality, corneal transparency degradation, corneal edema, vision decline or loss, eye dryness and pain resulting from corneal endothelial decompensation.

15. Use according to claim 11, wherein said medical instruments are graft, patch or kit.
